(19) United States
(12) Patent Application Publication
(10) Pub. No.: US 2006/0100612 A1
(43) Pub. Date: May 11, 2006

(54) Laser-based device for non-mechanical, three-dimensional trepanation during cornea transplants

(75) Inventors: Gerd van der Heyd, Adelsdorf (DE); Michael Harrer, Hessdorf (DE); Achim Langenbucher, Buckenhof (DE); Reinhold Frankenberger, Gerhardshofen (DE)

Correspondence Address: BROWDY AND NEIMARK, P.L.L.C.;624 NINTH STREET, NW, SUITE 300, WASHINGTON, DC, 20001-5303, US

(73) Assignee: Quintis GmbH, Erlangen (DE)

(21) Appl. No.: 10/525,391

(22) PCT Filed: Aug. 16, 2003
(86) PCT No.: PCT/EP03/09078

§ 371 Date: Feb. 22, 2005

(30) Foreign Application Priority Data
Aug. 20, 2002 (DE) 10237945.9

(51) Int. Cl. A61B 18/18

(52) U.S. Cl. 606/4

(57) ABSTRACT

A laser-based device for non-mechanical, three-dimensional trepanation during cornea transplants comprises
a computer-assisted control and regulation unit (4) provided with at least one control computer (5, 6, 7) and at least one display unit (8, 9), as well as
a laser source (2) for generating a working laser beam (3) as well as
a multi-sensor processing head (1) integrated into which are:
an axial beam positioning system (11) into which the working laser beam (3) can be coupled,
a focal point tracking unit (12) for the z-position displacement of the focal point (13) of the working laser beam (3)
an x-y-scanner unit (14, 15) for the x and y-position displacement of the working laser beam (3),
an eye position sensor unit (23, 24, 35, 36) for detection of the position of the eye, and
a plasma sensor unit (16, 25) for detection of the plasma glow that occurs during the cornea trepanation.

The invention relates to a laser-based device for non-mechanical, three-dimensional trepanation during cornea transplants. A device of this type is intended to serve particularly for cutting self-sealing, self-anchoring small tissue slices for cornea transplantation, as well as for the preparation of cornea lamellae adjoining the posterior cornea surface (PLK), the anterior surface (lamellar keratoplasty), or within the cornea.

Regarding the background of the invention, the current state of ophthalmic surgery technology for cornea transplantation shall be explained briefly in connection with devices for providing for donor-recipient corneas, as follows:

The classic implantation technique provides for a mechanical trepanation process by means of a Keratom or round scalpel. During the cornea transplant, a small round slice of approximately 7-8 mm diameter is removed from the donor and placed and sewn into the equivalent location at the recipient's.

The mechanical variant has the widest distribution, but it has the shortcoming that only circular cuts perpendicular to the tissue are possible and that pressure forces must be exerted during harvesting of the cornea slice which result in mechanical deformations and thus in irregular cuts. These pressure forces in combination with traction forces of the holding sutures while sewing in the transplant frequently lead to persistent tissue tensions and subsequently to optical distortions that are difficult to compensate for with glasses or contact lenses.

The device incorporates no sensor technology or position feedback. The quality of the harvested transplant with respect to an exactly defined and reproducible cutting geometry and smooth cutting edges depends entirely on the surgeon, so that a series of random influences therefore impacts the result.

Non-mechanical trepanation methods are laser-based and operate with an Excimer or Erbium:YAG laser, however their use is currently not as widespread. They prevent the mechanical deformation, however, there is a risk that the comparatively high-energy laser beam may heat the cutting area and result in thermal damage there. This method, too, allows for straight cuts to be made at nearly any angle to the surface. Undercuts cannot be generated with this system technology either.

These systems are usually provided with sensor technology and downstream image-processing tracking systems that register movements of the object being processed to a frequency of up to 200 Hz and that track the working position with a reaction time of greater than 5 ms. Lasers that are currently on the market can be adequately repositioned in this manner.

In the case of PLK methods, to remove the damaged lamella, a slice is cut from the patient's cornea on the posterior of the cornea comparable to the cornea transplant, and a posterior lamella is subsequently prepared from it. Afterwards a transplant is placed onto the rear of the slice in place of the removed volume element, sewn in, and the entire slice with the transplant is sewn back into the patient's wound.

Regarding the printed prior art, reference needs to be made to various publications. US 2001/0010003 A1, for example, reveals a method and apparatus for cornea surgery, wherein short laser pulses with shallow ablation depth are used. The apparatus in this context exhibits different basic components of processing systems for the cornea treatment, such as a central, computer-based control and regulation unit, an associated laser source and a beam positioning system for the working laser beam. Each pulse is directed into its desired position by means of a controllable laser-scanner system, wherein the laser pulses and energy deposited into the cornea surface are distributed in such a way that the surface roughness is controlled within a specific range. Additionally, a laser beam intensity sensor and a beam intensity adjustment means are provided so that a constant energy level is maintained throughout a surgery. The eye movement during the surgery is corrected for by means of a corresponding compensation in the beam position, for which a position detection system for the eye is provided.

The system according to the above printed publication exhibits the problem that no exact and sensitive monitoring of the cutting depth of the working laser beam takes place. This is not a highly relevant parameter for the purpose of the superficial cornea ablation on which the known surgical apparatus is chiefly based. In the case of the complete separation of the cornea, however, as it occurs during the trepanation, this problem does become acute.

Additionally it should be noted that while the printed prior art publications do show basic designs of laser-based ophthalmic surgical systems, these systems have so far been implemented in their complex form as laboratory set-ups on optical benches. Systems of this type are not suitable for widespread practical application.

Additional printed publications that show laser-based ophthalmic surgery systems are U.S. Pat. No. 6,325,792 B1 and U.S. Pat. No. 5,984,916 A.

Regarding the technological background, reference needs to be made to additional prior art. DE 199 32 477 C2, for example, shows a device for phototherapy in the eye, especially for photocoagulation of certain points in the background of the eye. In the process, the acoustic or optical signal that is caused by the change in the material as a result of the laser radiation is separated in a specific fashion from the so-called thermo-elastic signal, which contains only information regarding material properties. To generate measuring signals that can be evaluated, chemical reactions, ablation, fiber transitions, etc., and among others also plasma formation are mentioned.

EP 0 572 435 B1 reveals a device for sclerostomy ab externo wherein a laser beam is introduced into the eye via a light guide. The material that is located immediately in front of the end of the light guide evaporates during processing and forms a gas or plasma bubble. This bubble disintegrates after a certain amount of time and is replaced by new fluid or new material. The disintegration time of this bubble represents a discrimination criterion for whether the end of the light guide is located inside the chamber of the eye or not. This makes it possible to monitor the operation in the transitional layer region between tissue and fluid.

It is an object of the invention to improve a laser-based trepanation device in such a way that highly precise trepanation results are attainable in the cornea region with a compact, easy-to-manipulate surgical system. The invention is based especially on the object of developing a system technology with integrated sensor technology that permits the generation of three-dimensional cutting geometries whereby self-sealing and self-anchoring transplants can be inserted as optimally as possible.

This object is met according to the characterizing portion of claim 1 in such a way that, as the core piece of the laser-based trepanation device, a multi-sensor processing head is provided into which the relevant beam positioning system components and sensor technology units are integrated. The multi-sensor processing head accordingly comprises the following:
an axial beam guiding means into which the working laser beam can be coupled,
a focal point tracking unit for z-position adjustment of the focal point of the working laser beam,
an x-y scanner unit for x-y position adjustment of the working laser beam,
an eye-position sensor unit for detection of the position of the eye, and
a plasma sensor unit for detection of the plasma glow that occurs during cornea trepanation.

The subclaims characterize advantageous improvements of the trepanation device which, in order to avoid repetitions, will be described in more detail with their corresponding functionalities and advantages based on the description of the example embodiment.

In summary it may be stated that the inventive trepanation device incorporates a laser-based processing head, which may be equipped with sensors for the positional detection of the object being processed, for distance measurements to the object, plasma and focal point position detection, laser output regulation, as well as multiple linear and tilting axes, and thus permits a highly precise three-dimensional trepanation of tissues with position feedback. With the sensor head it is possible to generate perfectly fitting undercuts (lock-and-key principle) both in the recipient and donor tissue (especially recipient and donor corneas) which, through their geometric design or the support of the eye pressure acting from inside, have a self-sealing function. The donor cornea can also be anchored in the recipient cornea in such a way that a subsequent sewing-in of the donor slice becomes necessary only to a limited extent or is eliminated altogether. Additionally it is possible, with a focussing on the posterior of the cornea and focal point tracking over the cutting profile, to remove a damaged region or volume element along a flat surface. The separated volume element can be removed through a cut made in the dermis and a homogenous or artificial volume element can simultaneously be inserted and integrated in a self-adhering manner through this cut.

Additional characteristics, advantages and details of the invention will become apparent from the following description in which an preferred embodiment will be explained in more detail based on the appended drawing, in which:

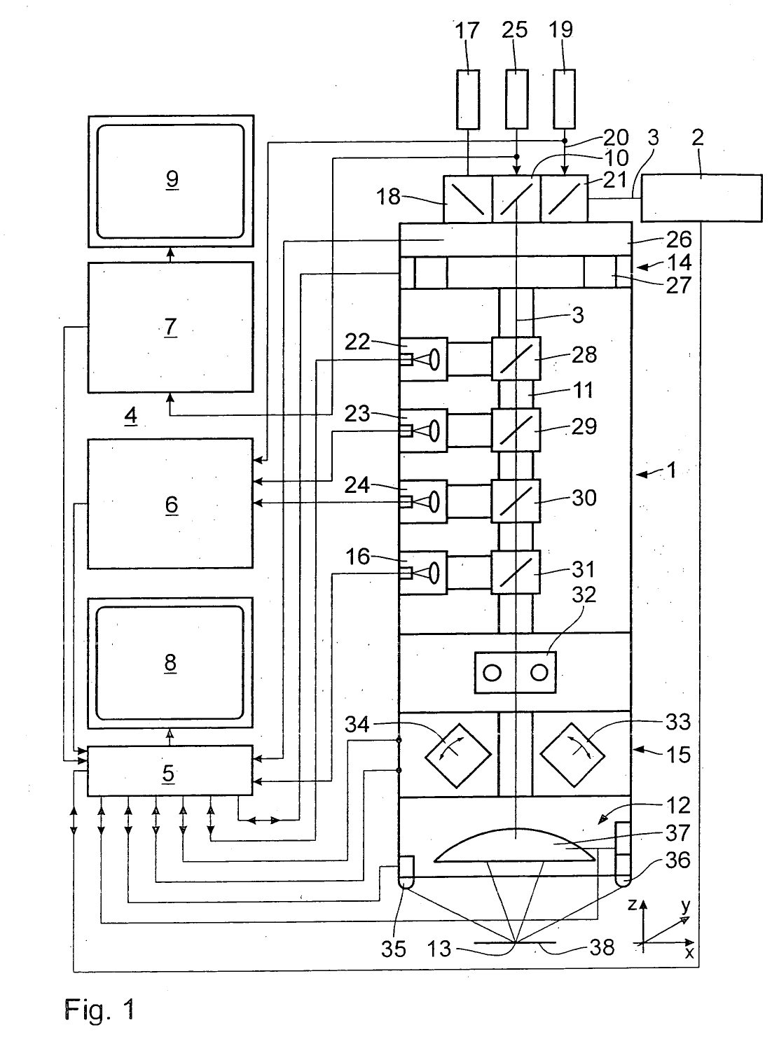

FIG. 1 shows a schematic system illustration of a laser-based trepanation device,

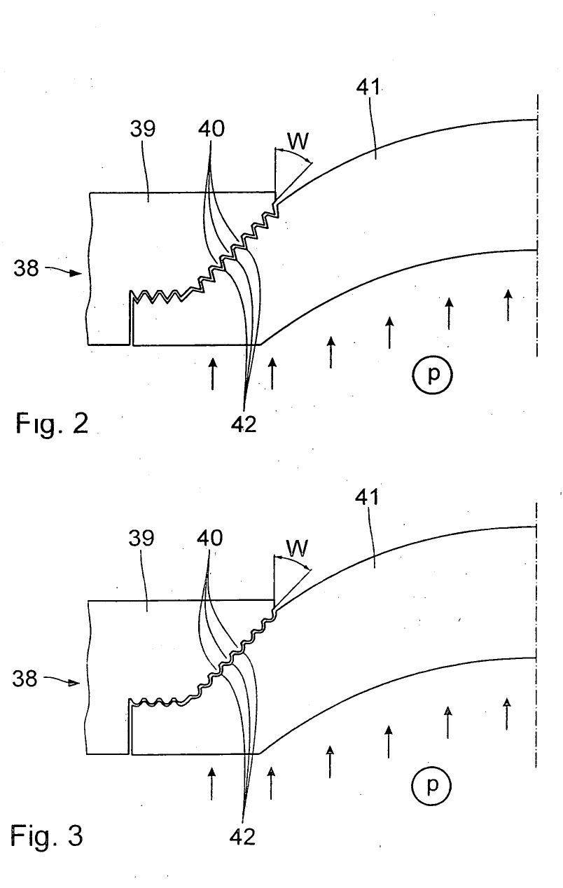

FIGS. 2 and 3 show enlarged schematic sections through a recipient/donor cornea in a first application,

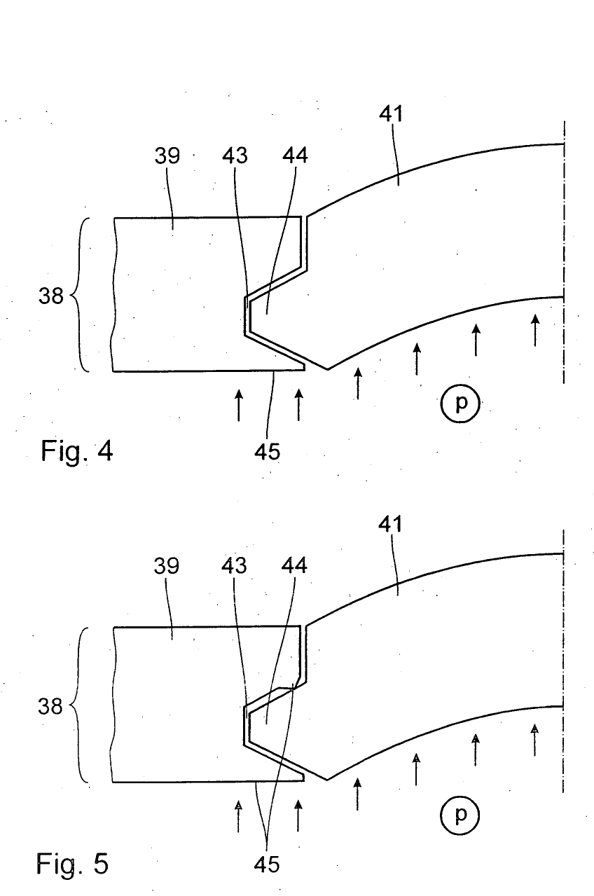

FIGS. 4 and 5 show schematic sections through a recipient/donor cornea in a second application,

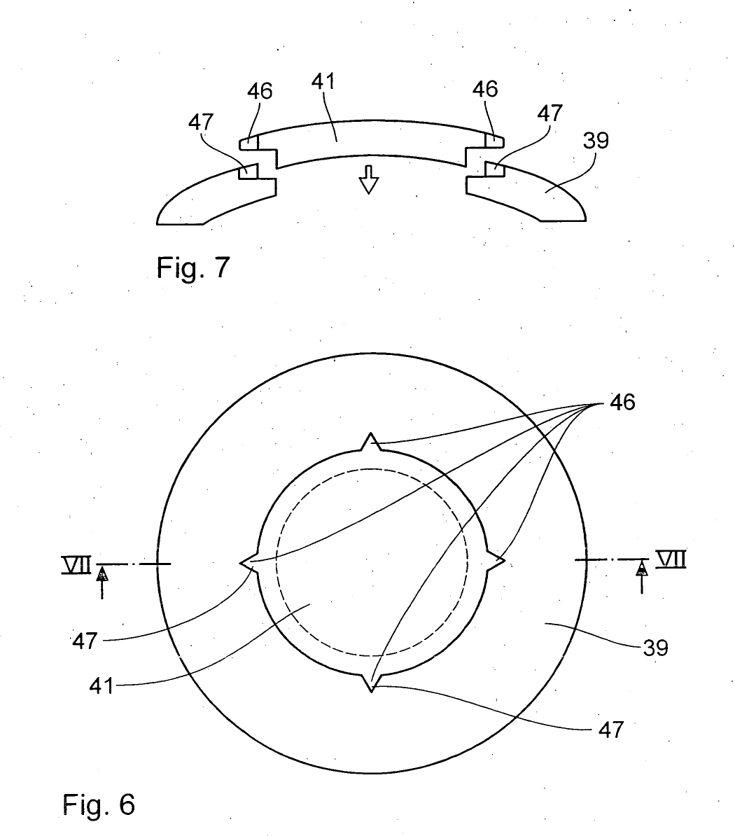

FIG. 6 shows a top view of a recipient/donor cornea in a third application, and

FIG. 7 shows a radial section through the cornea along the section line VII-VII according to FIG. 6.

The overall system of the laser-based trepanation device shown in FIG. 1 has, as the core element, a multi-sensor processing head, which is denoted in its entirety with the numeral 1, to which a laser source 2 is assigned for generating a working laser beam 3, and a control and regulation unit, which is denoted in its entirety with the numeral 4. The latter comprises—as will be explained in more detail below—three control computers 5, 6, 7, as well as two displays 8, 9, e.g., in the form of conventional monitors.

The multi-sensor processing head will be explained in more detail below. The working laser beam 3 is coupled via a deflection prism 10 into the beam guiding system 11 that defines the optical axis of the multi-sensor processing head 1. A focal point tracking unit 12, marks the end of the beam guiding system 11 opposite the deflection prism 10, which focal point tracking unit 12 adjusts the focal point 13 of the working laser beam 3 in the thus defined z-position along the z-direction extending in the direction of the beam guiding system 11.

The x-y position adjustment of the working laser beam 3 is carried out by a two-stage x-y scanner unit that is composed of a rough-adjustment unit 14 at the coupling-in end of the beam guiding system 11 and a fine-adjustment unit 15 on the end of the beam guiding system 11 closest to the object being treated.

The multi-sensor processing head has assigned to it additional illuminating units, namely first of all an adjusting laser 17 that is coupled coaxially into the optical axis of the beam guiding system 11 via a deflection prism 18 that is positionable in x-y-z direction. The adjusting laser 17 emits radiation in a wavelength spectrum that is visible to the eye and serves the surgeon for the rough positioning of the multi-sensor processing head 1. The adjusting units that are used for the prism 18 have an operating range of 5 mm with a positioning accuracy of ±0.01 mm.

Additionally, an infrared illuminating unit 19 is provided whose infrared beam 20 is also coupled into the beam guiding system 11 “on axis”via a deflection prism 21 that is adjustable in x-y-z direction. It serves for a high-contrast illumination of the pupil, which brings with it advantages that will be explained further below. For the IR illuminating unit 19, IR laser diodes may be used, for example, wherein the variation of the illumination intensity can be implemented via a current or voltage regulator.

Also integrated into the multi-sensor processing head 1 are various camera and sensor units, which, for the sake of clarity, will only be listed at this point and explained in more detail below. Provided downstream of the rough-positioning unit 14 is a laser performance sensor 22. It is followed in the beam guiding system 11 by two CCD line scan cameras 23, 24 that form part of an eye position sensor unit. These CCD line scan cameras 23, 24 determine on-line the position of the pupil or of a marker on the cornea or dermis of the eye that is specifically applied for the procedure. They consist of two IR-sensitive high-speed line scan cameras whose line orientation is arranged orthogonal to one another and coupled into the beam path. The cameras have a resolution of 8192 pixels on the approximately 20-25 mm large image section of the eye. From this results a position inaccuracy of less than 10 μm. The cameras provide more than 250 lines per second, which are evaluated in real time, so that all spontaneous eye movements—even fast saccades during surgery—are registered. The data are routed via RS422 interfaces or CameraLink interfaces to the computer unit 6, which functions as the positioning computer.

The data from the cameras are evaluated via this computer 6 and the position of the eye is extracted in the x-y-plane with modern methods of digital image analysis. In the process, the comparatively strong contrast between the iris and pupil is utilized that is generated by the IR illuminating unit 19. Through backscatter of the IR illumination on the retina the pupil appears clearly lighter and sharply delineated relative to the iris in the line data of the cameras 23, 24. Filters, which are tuned to the IR illumination, in front of the objectives of the line scan cameras 23, 24 prevent the influence of ambient light on the measuring results and ensure the adequate contrast between iris and pupil for a reliable detection of the structures. The position data that have been determined in this manner are transmitted to the computer control and used in the case of a position change for correction of the beam position.

In lieu of the previously mentioned plasma sensor 16, or in addition to it, a CCD area scan camera 25 is provided in order to detect and analyze by means of modern digital image processing the quality of the plasma. The plasma of the above-described laser ignites when coupled into tissue, but not in water, specifically not in the aqueous humor behind the endothelium of the cornea. This provides an opportunity to verify whether the focal point 13 of the working laser beam 3 is localized in the anterior chamber or in the cornea tissue. This is important in order to monitor the complete separation of the cornea lamellae during the penetrating cornea trepanation. With the CCD area scan camera 25, the glow of the plasma is detected with position resolution. The comparison of the image taken with the camera 25 with and without plasma glow permits conclusions as to whether the tissue was completely separated. If the trepanation was not complete—i.e., the plasma glow is still visible—the laser beam again couples in at this position and separates the remaining tissue remnants. As soon as no more plasma glow can be detected, the tissue is completely separated and the cutting process is stopped.

The camera 25 is able to deliver more than 250 images per second at a resolution of 768×560 pixels and transmits the obtained image data to the computer 7, which, as the control computer, performs the evaluation and controls the laser in accordance with the pupil contour and the data obtained from the plasma detection. Located in front of the camera is a filter that is tuned to the plasma glow of cornea tissue.

If position resolution of the plasma glow is not required, only the plasma sensor 16 needs to be used.

Control of the working laser beam 3 in its x-y-position occurs—as already outlined above—on the one hand by means of the rough-adjusting unit 14, which is comprised of an x-axis prepositioning unit 26 and a y-axis prepositioning unit 27. The two prepositioning units 26, 27 may be deflection mirrors that are mounted on the corresponding axes, in such a way that the two prepositioning units can be set up of two linear axes, one linear and one tilting axis, two tilting axes or also of two rotatory axes. The positioning accuracy of the axes is approximately ±0.1 mm. After the rough adjustment, which may take place with the aid of the beam of the adjusting laser 17 entered into the beam guidance 1 1, these axes are locked in order to rule out any unintentional adjustment during the fine adjustment or eye measurement.

The image data of the CCD area scan camera 25 are used in other respects to determine the contour of the pupil. At the beginning of a trepanation process, the contour of the pupil is determined with the aid of rim detection filters in the computer 7. The contour data enter into the calculation of the position of the pupil in the x-y plane in order to compensate for deviations from the ideal-circular shape of the pupil.

The above mentioned laser output sensor 22 measures the laser output during the processing to achieve an optimal processing result and thus permits a targeted output control. For this purpose approximately 1 to 5% of the laser output is coupled out via a pick-off lens 28 that is installed on axis in the beam guiding system 11, and detected with the sensor 22. The signal that is obtained in this manner is utilized as a positioning value for a real time output control of the working laser beam 3, as well as for statistical purposes. The laser output sensor 22 is coupled for this purpose with the central control computer 5 via an appropriate interface.

The above-mentioned CCD line scan cameras 23, 24 and the facultative plasma serisor 16 are also supplied, via pick-off lenses 29 through 31, with the corresponding signals from the beam guiding system 11.

In the further course of the beam positioning system in the direction toward the processing location, a surgery microscope 32 is coupled into the beam guiding system 11, whereby the trepanation process can be observed and monitored by the surgeon in the usual manner.

The previously mentioned fine adjusting unit 15 may, in principle, use nesting, uni-axial or multi-axial rotatory axes (e.g., galvanic scanners) with limited dynamics or piezo-actuators (linear axes with translation or tilting axes) as systems with extremely high dynamics or also combinations of the two for the beam deflection with mirrors or prisms. Since only a small working area must be covered for the inventive applications, mirror-tilting systems 33, 34 with piezo-drive are used that are coupled into the beam path, which deflect the beam 3 for fine processing in the x-y plane. Stacked piezo actuators provide the necessary tilting angle of ±2 degrees, which is comparatively high for piezo actuators. An additional criterion is the high resonance frequency of over 1 kHz, as well as the very high positioning accuracy of 0.1% at a reproducibility of 0.04% and an extremely high linearity of the tilting axes over the positioning range.

The multi-sensor processing head 1 is additionally provided at its lower end with two laser distance sensors 35, 36, one of which determines the distance to the center of the cornea, whereas the other measures the distance of a point in the rim region of the cornea. The laser distance sensors 35, 36 operate, e.g., according to the triangulation principle with a weak laser beam in the near infrared range (approximately 810-1200 nm). Both sensors 35, 36 provide, with an output sequence frequency of 1 kHz, measuring values for the distance to the cornea. From these two distance values the position of the eye to the processing head 1 is determined with the aid of the central control computer 5. The accuracy of the sensors is approximately 10 μm. With the aid of the measuring values in the x-y plane from the positioning system 23, 24 and the measuring values of the two distance sensors 35, 36, the positioning computer 6 determines the position of the eye in three directions in space. In the process, previously determined data regarding the cornea topography and cornea thickness are relied upon. If no topography data are available, a spherical surface is assumed for the geometry of the cornea rim areas for modeling purposes.

The central computer 5 implements the focal point tracking of the system. In principle, two system techniques can be applied, namely a focal point tracking by means of adaptive optics or by displacement of a telecentric focussing lens. The adaptive optics may be set up as a transmissive element (by means of lenses) or as a reflective element (by means of mirrors). Both systems are characterized in that the lens or mirror curvature is altered by means of pressure exerted onto the lens or mirror, which is accompanied by a displacement of the focal point. The invention preferably employs the focal point tracking by means of displacement of a telecentric focussing lens 37. In the process, the lens 37 which is disposed displaceable in the z-plane and has a fixed focus in dependence upon the position of the mirror tilt systems 33, 34 of the fine adjusting unit 15, is displaced in such a way that predefined profiles in the space are scanned with the focal point of the laser source.

The control of the focussing lens as well as of the tilting systems 34, 34 may be provided with position feedback coupling outputs that are not shown in detail for position control of these components.

Additionally, the position of the eye that has been obtained with the aid of the positioning system 23, 24 and the distance sensors 35, 36 enters into the control process in a correcting manner. The positions of each mirror axis of the scanning units are fed back during the focal point tracking, monitored by the central control computer 5, and optionally corrected.

The displays 8, 9 that were mentioned at the beginning consist of a monitor 8, which is connected to the central control computer 5 and which displays planning, monitoring and simulation images and data.

The second display 9 is connected to the control computer 7 that is coupled with the CCD area scan camera 25 and can display a live image and/or the eye position.

The explained trepanation system makes it possible to remove a posterior lamella of the cornea without temporarily completely removing a slice of the cornea from the patient. Only one additional cut is required in the dermis of the patient's eye, comparable to a cataract access, through which the lamella can be removed and through which the implant can be inserted and adjusted.

This technology in particular requires highly precise sensors and laser control. In order to be able to cut lamellae in different thicknessses, the focal point position of the laser must be exactly defined and controlled and have an extremely short shaft length.

In summary, it is not possible with any of the systems according to the prior art to cut a self-sealing, self-anchoring structure in corneas in such a way that the subsequent sewing-in of the transplant can be significantly reduced or completely eliminated. Additionally, it is not possible with any of the earlier systems to perform, with reasonable effort, lamellar surgery on the posterior of the cornea without damaging the front of the cornea.

The application of the inventive trepanation device shall be explained in more detail based on FIG. 2 through 7. FIGS. 2 and 3, for example, show radial partial sections through the cornea area 38 of the eye, wherein the remaining donor cornea 39 has along its rim saw-tooth shaped (FIG. 2) or bulge-like (FIG. 3) raised areas 40, which find corresponding negative shaped recesses 42 in the donor cornea 41. The entire structure extends at an angle w of approximately 45° through the thickness of the cornea 38, as indicated in both figures, so that the denticulations between the raised areas 40 and recessed areas 42 are pushed into one another by the internal pressure of the eye p (see arrows in FIGS. 2 and 3) and an increased sealing effect along the type of a flat seal with a simultaneous associated self-anchoring are attained in the process.

FIGS. 4 and 5 show sectional views analogous to FIGS. 2 and 3 wherein a circumferential larger groove 43 in the recipient cornea 39 receives a corresponding connecting projection 44 on the donor cornea 41. Provided on the groove are sealing lips 45, which again provide for a seal through the internal eye pressure p.

FIGS. 6 and 7, in turn, show a self-anchoring geometry of the implant in the form of the donor cornea 41. For this purpose, an interlocking, undercut connection is created between the recipient and donor cornea 39, 41, namely by establishing a radial denticulation or with radial connecting projections 46 and corresponding grooves 47 on the donor cornea 41 and recipient cornea 39. These connecting projections 46 and grooves 47 also assume the function of a marker for the rotational position of the implant 41 in the recipient cornea 39.

1. A laser-based device for non-mechanical, three-dimensional trepanation during cornea transplants, comprising
a computer-assisted control and regulation unit (4) provided with at least one control computer (5, 6, 7) and at least one display unit (8, 9),
a laser source (2) for generating a working laser beam (3), and
a multi-sensor processing head (1) integrated into which are:
an axial beam guiding system (11) into which the working laser beam (3) is coupled,
a focal point tracking unit (12) for a z-position displacement of a focal point (13) of the working laser beam (3),
an x-y-scanner unit (14, 15) for a x- and y-position displacement of the working laser beam (3),
an eye position sensor unit (23, 24, 35, 36) for detection of a position of the eye, and
a plasma sensor unit (16, 25) for detection of a plasma glow that occurs during the cornea trepanation, which plasma sensor unit (16, 25) is coupled with the control computer (5, 6, 7), which controls the laser on the basis of data derived by said plasma detection.

2. A trepanation device according to claim 1, comprising an adjusting laser (17) whose visible adjustment beam is coupled into the axial beam guiding system (11) via a deflection prism (18) that is positionable in x-y-z direction.

3. A trepanation device according to claim 1, comprising an infrared illuminating unit (19) whose infrared beam (20) is coupled into the axial beam guiding system (11) via a deflection prism (21) that is positionable in x-y-z direction.

4. A trepanation device according to claim 1, wherein the focal point tracking unit (12) comprises one of adaptive optics and a displaceable telecentric focussing lens (37).

5. A trepanation device according to claim 1, wherein the x-y scanner unit comprises a rough adjustment unit (14) with two adjusting axes (26, 27) and a fine adjusting unit (15) preferably with piezo-driven tilting mirrors (33, 34).

6. A trepanation device according to claim 1, wherein the x-y scanner unit (14, 15) and the focal point tracking unit (12) comprise position feedback outputs, which are coupled with the control and regulation unit (4) for controlling the actual x-y-z position of the focal point (13) of the working laser beam (3).

7. A trepanation device according to claim 1, wherein the eye position sensor unit comprises two CCD line scan cameras (23, 24) that are orthogonal in their line orientation.

8. A trepanation device according to claim 1, wherein the eye position sensor unit comprises two laser distance sensors (35, 36), one of which determines its distance to the center of the cornea being treated and the other determines its distance to a rim point of the cornea.

9. A trepanation device according to claim 1, wherein the plasma sensor unit is formed by one of a CCD area scan camera (25) for position-resolved detection of the plasma glow and a plasma sensor (16).

10. A trepanation device according to claim 9, wherein the image data of the CCD area scan camera (25) is used for determining the pupil contour of the eye being treated.

11. A trepanation device according to claim 1, wherein a laser output sensor (22) in the multi-sensor processing head (1).

12. A trepanation device according to claim 1, wherein a surgery microscope (32) is integrated into the multi-sensor processing head (1).

13. A trepanation device according to claim 1, wherein the control and regulation unit (4) comprises a central control computer (5), a positioning computer (6) that is coupled with the CCD line scan cameras (23, 24) and with the infrared illuminating unit (19), and a control computer (7) that is coupled with the CCD area scan camera (25).

14. A trepanation device according to claim 1, wherein the display unit comprises multiple displays (8, 9) for displaying a real-time image of the eye being treated with the plasma glow and displaying planning, monitoring and simulation images and data.
